(19) United States
(12) Patent Application Publication
(10) Pub. No.: US 2026/0324965 A1
(43) Pub. Date: Oct. 22, 2026

(54) TWO BUTTON BRONCHOSCOPY DEVICE WITH RESPIRATORY SUPPORT AND METHODS OF USE THEREOF

(71) Applicant: Carilion Clinic, Roanoke, VA (US)

(72) Inventors: Edmundo R. Rubio, Roanoke, VA (US); David Ryan King, Roanoke, VA (US); Robert Fippinger, Roanoke, VA (US)

(21) Appl. No.: 19/650,459

(22) Filed: Apr. 17, 2026

Related U.S. Application Data
(60) Provisional application No. 63/791,009, filed on Apr. 18, 2025.

(51) Int. Cl. A61B 1/267; A61B 1/00; A61B 1/005; A61B 1/015; A61B 1/07

(52) U.S. Cl. CPC A61B 1/2676; A61B 1/00121; A61B 1/0052; A61B 1/015; A61B 1/07

(57) ABSTRACT

A bronchoscope device with a handle that includes a switch for controlling whether pressurized gas, for example medical grade oxygen, or a suction is applied to a fluid conduit within the insertion tube of the bronchoscope. Also described are methods of using the bronchoscope device to restore a patient's oxygen saturation without removing the insertion tube.

### BACKGROUND

Bronchoscopy is a surgical technique that allows a doctor to view the airways of a patient using an endoscope device that is particularly adapted to insertion into the airways of the patient, otherwise known as a bronchoscope. The bronchoscope typically includes an insertion tube that includes a suction channel and one or more flexible fiber optic instruments. The fiber optic instruments can include a light and a camera. The device is typically inserted through the patient's mouth or nasal cavity and allows the doctor to explore the patient's trachea, main stem bronchi, and some of the small bronchi. It is frequently used to remove small objects that have been inhaled, often by children. It is also often used to take biopsy samples of lesions found in a patient's lung.

As shown in FIG. 1, a bronchoscope 100 is being used on the lung 106 of a patient 102. The bronchoscope 100 includes a handle 102 and an insertion tube 104. The tip 106 of the insertion tube extends into the small bronchi 108. In FIG. 2, a convention endoscope 201 as described in U.S. Patent No. 12,245,752 is shown. The endoscope is disposable, and not intended to be cleaned and reused. The endoscope 201 comprises an elongated insertion tube 203. The insertion tube 203 is suitable for insertion into a lung of a body through a mouth. The body can be an artificial or natural body, for instance a human or animal body. At the proximal end 203 a of the insertion tube 203 an operating handle 202 is arranged. The operating handle 202 has a control lever 221 for maneuvering an articulated tip part 205 at the distal end 203 b of the insertion tube 203 by means of a steering wire (not shown) running through a flexible tube 209. A camera assembly (not shown) is positioned in the tip part 205 and is configured to transmit an image signal through a monitor cable (not shown) of the endoscope 201 to a monitor (not shown). The endoscope also includes a button that is configured to turn on the suction and apply suction to the tip part 205.

There is a need to improve the bronchoscope and methods of using the scope to make it more versatile and allow for procedures to be performed on patients experiencing desaturation of oxygen.

### SUMMARY OF THE INVENTION

In one aspect, the present invention relates to a bronchoscope device comprising an insertion tube, the insertion tube comprising a fluid channel and a fiber optic element; and a device handle connected to the insertion tube, the handle comprising: a pressurized gas inlet; a pressurized gas button, the pressurized gas button has a pressurized gas off-state wherein pressurized gas is not supplied to the insertion tube and a pressurized gas on-state wherein pressurized gas is supplied to the insertion tube, the pressurized gas button being naturally bias in the pressurized gas off-state; a suction inlet; and a suction button, the suction button has a suction off-state wherein suction is not supplied to the insertion tube and a suction on-state wherein suction is supplied to the insertion tube, the suction button being naturally bias in the suction off-state. The user of the bronchoscope can supply: (1) pressurized gas to the insertion tube by pressing the pressurized gas button, and (2) suction to the insertion tube by pressing the suction button.

In one aspect, the pressurized gas can be medical grade oxygen or filtered air. The device can be used to deliver pressurized gas comprising oxygen through the insertion tube when the patient's SpO₂ falls below 90%.

In one embodiment, the buttons have a natural bias to be in the off-state. For example, the pressurized gas button and/or the suction button may include a spring to bias the pressurized gas button in the pressurized gas off-state. The pressurized gas inlet may be attached directly to the pressurized gas button, or alternatively the pressurized gas inlet may be attached directly to the side of the device handle.

The invention also relates to methods of operating the bronchoscope device wherein the user of the bronchoscope can use the selector to cause either suction or pressurized gas to be applied within the fluid channel of the insertion tube of the bronchoscope. The method may entail delivering oxygen through the insertion tube when the patient's SpO₂ falls below 90%. This may allow continuing an operation whereby the insertion tube is not removed from the patient while the pressurized gas is being delivered. In one aspect, the bronchoscopy procedure is continued without removing the insertion tube after the patient's oxygen saturation is restored. In addition, the oxygen may also be used to clear plugs/blocks at the distal end of the inlet tube that occur when objects for suction are too large for the lumen or to clear debris off of the fiber optic camera. This aspect can increase the efficiency of the device since it can reduce the amount of time procedures are paused. In one aspect, the bronchoscopy procedure is continued without removing the insertion tube after the patient's oxygen saturation is restored.

### BRIEF DESCRIPTION OF THE DRAWINGS

FIG. 1 shows a side view of a conventional bronchoscope delivered to a bronchos of the patient;

FIG. 2 shows a conventional bronchoscope;

FIG. 3A shows a view of a bronchoscope according to an embodiment of the invention;

FIG. 3B shows a first inside view of the bronchoscope according to an embodiment of the invention;

FIG. 3C shows a second inside view of the bronchoscope according to an embodiment of the invention;

FIG. 4A shows a cross-sectional side view of a two button bronchoscope according to an embodiment of the invention.

FIG. 4B shows a top down view of a two button bronchoscope according to an embodiment of the invention.

FIG. 4C shows a side view of the bronchoscope according to an embodiment of the invention.

FIG. 4D shows a top down view of the bronchoscope according to an embodiment of the invention.

FIG. 4E shows a front cross-sectional view of the bronchoscope according to an embodiment of the invention.

FIG. 4F shows a perspective view of the bronchoscope according to an embodiment of the invention.

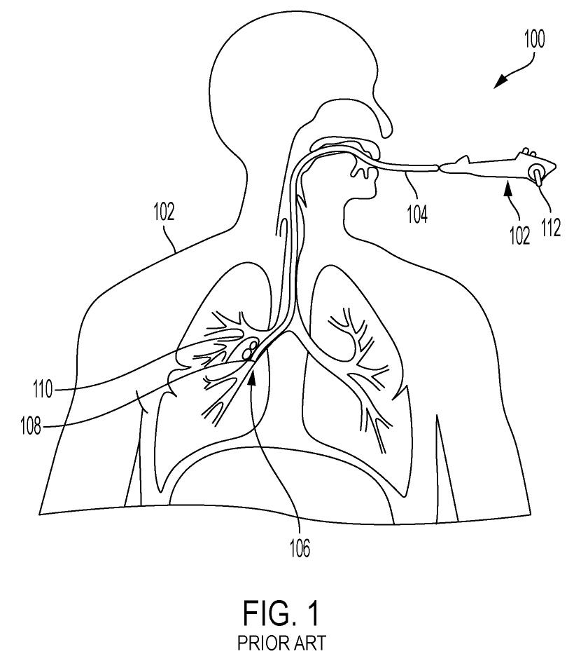

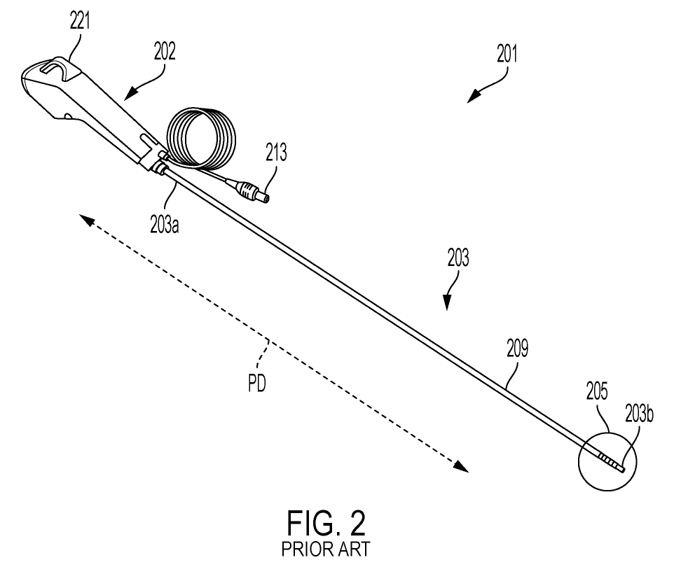

### DETAILED DESCRIPTION

The present inventors have found that there is a need for a treating physician to conveniently supply oxygen during the course of a bronchoscopy operations where a patient's oxygen saturation falls below 90% without removing the insertion tube of the bronchoscopy device. In addition, the oxygen may also be used to clear plugs/blocks at the distal end of the inlet tube that occur when objects for suction are too large for the lumen or to clear debris off of the fiber optic camera. This aspect can increase the efficiency of the device since it can reduce the amount of time procedures are paused. In the case of non-disposable bronchoscopes, the suction (e.g., vacuum) supply button is integrated with a valve having a suction inlet that includes barbs for connecting to a suction line. The suction button can then be replaced as necessary. The provision of oxygen is controlled by a switching element such as a button that is present on the handle of the bronchoscope device.

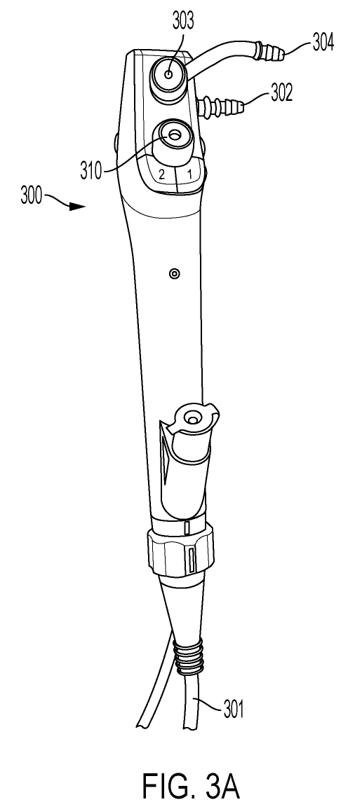

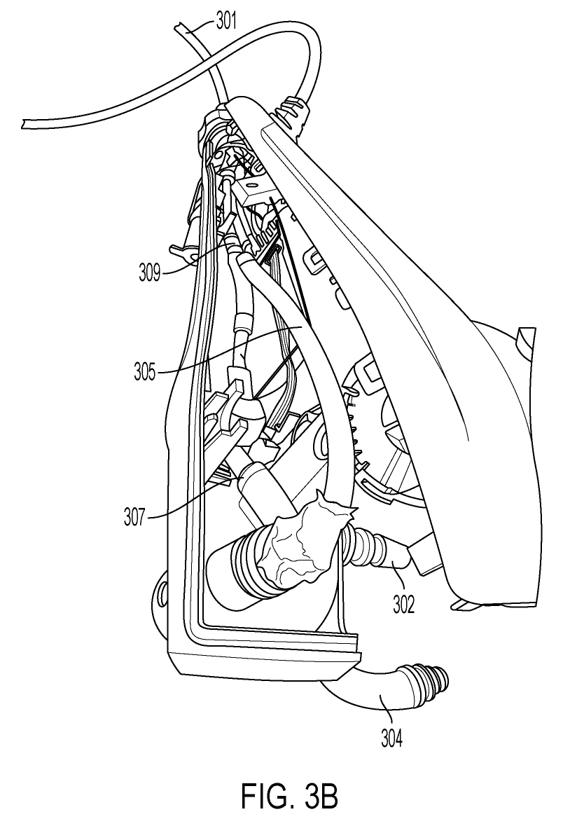

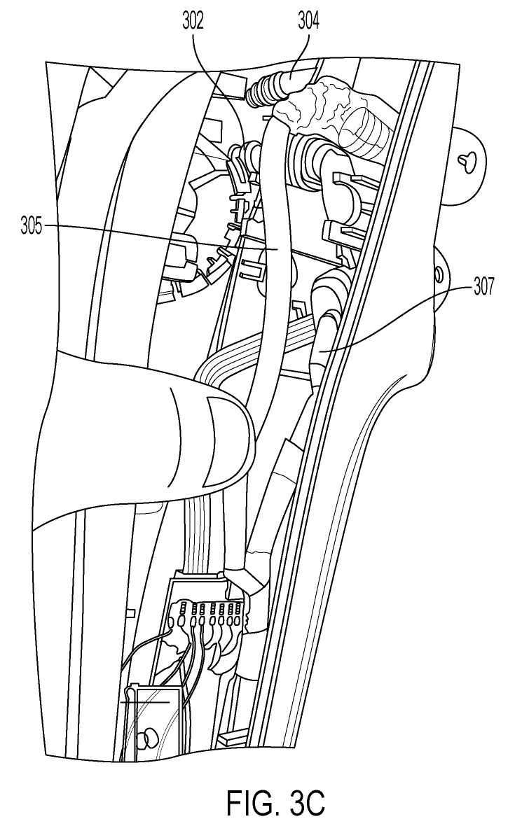

FIG. 3A shows a view of a bronchoscope device according to an aspect of the invention. A handle 300 is provided that includes an insertion tube 301, a suction inlet 302 and a button 303. In this embodiment, the suction inlet 302 is a repurposed suction inlet that is typically used with non-disposable bronchoscopy devices, e.g., as shown in U.S. Pat. No. D871,574 S. The suction inlet 302 provides suction to an internal fluid channel of the insertion tube 301. A pressurized gas inlet 304 is provided for supplying oxygen (or an oxygen containing gas) to the fluid channel of the insertion tube 301.

As shown in FIGS. 3B-3C, the pressurized gas inlet 304 supplies the pressurized gas to the first fluid conduit 305 within the handle of the bronchoscope device. The suction inlet 302 is connected to a second fluid conduit 307 within the handle of the bronchoscope device. A third fluid conduit 309 in communication with the fluid channel of the insertion tube 301. A t-connector 306 merges the third fluid conduit 309 with both the first fluid conduit 305 and the second fluid conduit 307.

The device shown in FIG. 3A includes button 303 which can be used to switch between the conventional supply of suction and suction to the internal fluid channel of the insertion tube 301. Other mechanisms for switching between the suction and provision of oxygen may be utilized. Preferably, the control mechanism is located within the handle of the bronchoscope device. Notably, the device includes a button 310 which may be non-functional, or alternatively can be provided with a desired function. For example, in one embodiment the button 310 could be used as an alternative way to turn off either suction or pressurized air

In one aspect, the invention relates to methods of bronchoscopy using the devices described herein. In this method, the operator of the device may supply oxygen-containing pressurized gas when a patient's oxygen desaturation SpO₂ falls below a predefined cutoff, typically 90%. By allowing oxygen to pass through a fluid channel of the insertion tube, the patient's lungs are supplied with oxygen that can allow the patient to recover to a desired level of oxygen saturation. Once the desired level of oxygen saturation is achieved, the bronchoscope device may be switched back to provide suction in the fluid channel of the insertion tube. This can allow the procedure to continue and restoration of the patient's oxygen saturation levels without removing the insertion tube.

In one aspect, the present invention allows for placement of the gas inlets on the same side of the device (i.e., both point left or right giving a “U” shape instead of left and right giving a “T” shape). The inlets being on the same side of the device is important to the useability of the device because it allows oxygen and suction hoses to be positioned on one side of the patient. This avoid the problem of tubes drawing across the patient/provider and reducing the mobility and flexibility with which the provider can position and remove the device.

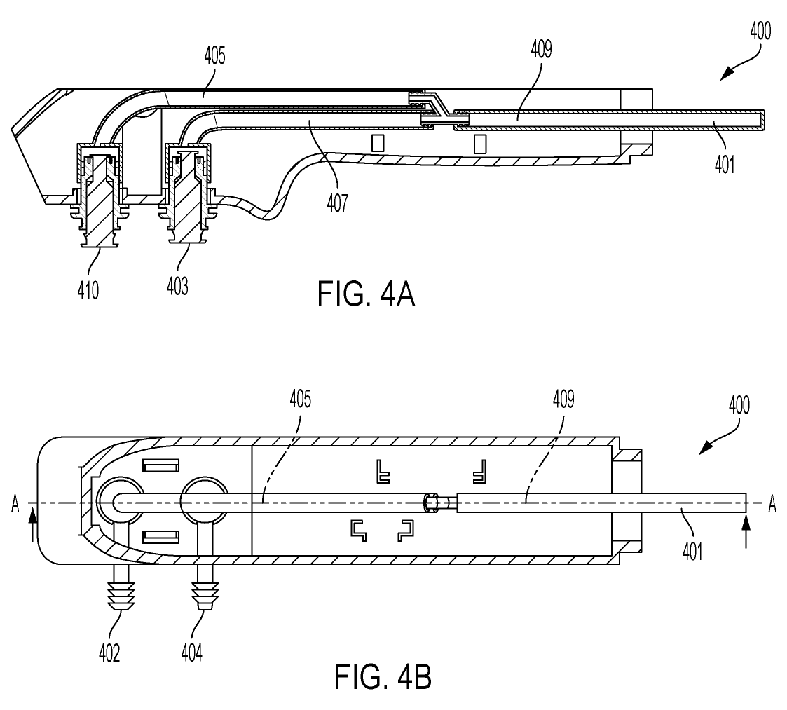

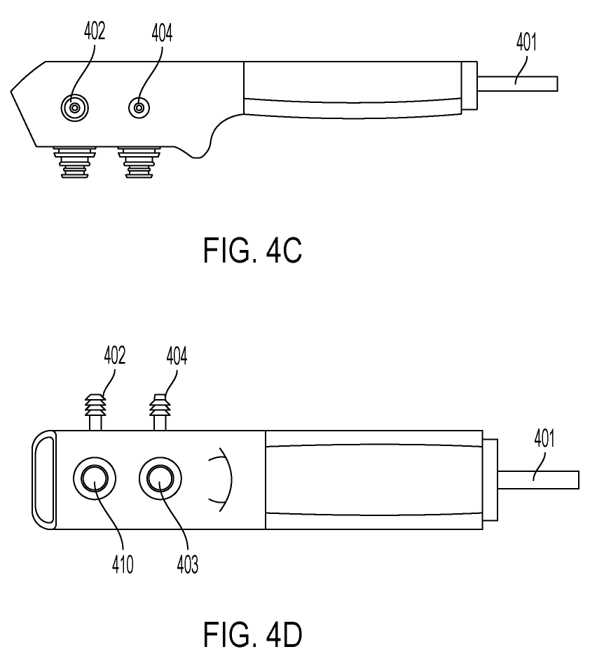

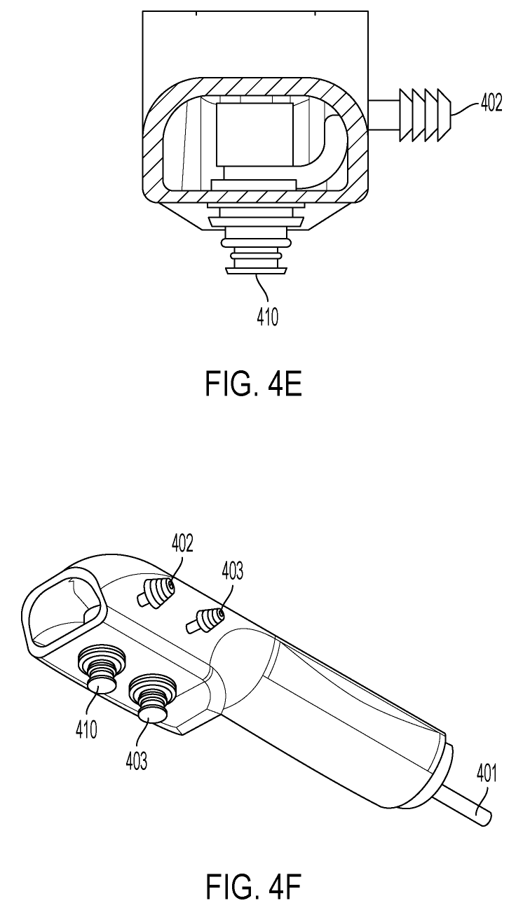

FIGS. 4A-4F show an example of configuration in another embodiment of a bronchoscope 400. FIG. 4A shows a side cross section of the bronchoscope 400. In this case, a suction inlet 402 is provided adjacent a button 410 for controlling suction. The suction inlet may have barbs that are used to facilitate connection of the suction inlet to a suction tube that may have an inside diameter of 7 mm. The suction inlet barbs may have successive increasing diameters of, for example, 8, 9, 10, and 11 mm. A pressurized gas inlet 404 is provided adjacent a button 403 for applying pressurized gas, e.g., oxygen. The gas inlet 404 is for supplying oxygen (or an oxygen containing gas) to the fluid channel of the insertion tube 401. The pressurized gas inlet may have barbs that are used to facilitate connection of the pressurized gas inlet to an oxygen tube that may have an inside diameter of 5 mm. The suction inlet barbs may have successive increasing diameters of, for example, 6, 7, and 8 mm.

When neither button 403 or 410 is depressed, there is neither a suction or oxygen supplied to the insertion tube 401. When the suction button 410 is depressed, suction is applied to the suction conduit 405, and then to the insertion tube 401. When the oxygen button 403 is depressed, oxygen is supplied via the pressurized gas supply inlet 404 to the oxygen supply conduit 407 and then to the insertion tube 401. It should be appreciated that the insertion tube and other aspects of a conventional bronchoscope device are not shown. FIG. 4B shows a top down view. FIG. 4C shows a side view showing the suction inlet 402 and pressurized gas inlet 404. FIG. 4D is top down view of the showing the suction inlet 402 and pressurized gas inlet 404 and the suction button 410 and pressurized gas inlet 403. FIG. 4E shows the view down the length of the bronchoscope with suction button 410 and suction inlet 402. FIG. 4F shows a perspective view of the bronchoscope.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A bronchoscope device comprising:
an insertion tube, the insertion tube comprising a fluid channel and a fiber optic element;
a device handle connected to the insertion tube, the handle comprising:
a pressurized gas inlet;
a pressurized gas button, the pressurized gas button has a pressurized gas off-state wherein pressurized gas is not supplied to the insertion tube and a pressurized gas on-state wherein pressurized gas is supplied to the insertion tube, the pressurized gas button being naturally bias in the pressurized gas off-state;
a suction inlet; and
a suction button, the suction button has a suction off-state wherein suction is not supplied to the insertion tube and a suction on-state wherein suction is supplied to the insertion tube, the suction button being naturally bias in the suction off-state;
wherein the user of the bronchoscope can supply: (1) pressurized gas to the insertion tube by pressing the pressurized gas button, and (2) suction to the insertion tube by pressing the suction button.

2. The bronchoscope device of claim 1, wherein the pressurized gas is medical grade oxygen.

3. The bronchoscope device of claim 1, wherein the pressurized gas inlet and the suction inlet are on the same side of the device.

4. The bronchoscope device of claim 1, wherein the bronchoscope device is configured to deliver pressurized gas comprising oxygen through the insertion tube when the patient's SpO₂ falls below 90%.

5. The bronchoscope device of claim 1, wherein the pressurized gas button comprises a spring to bias the pressurized gas button in the pressurized gas off-state.

6. The bronchoscope device of claim 1, wherein the suction button comprises a spring to bias the suction button in the suction off-state.

7. The bronchoscope device of claim 5, wherein the pressurized gas inlet is attached to the pressurized gas button.

8. The bronchoscope device of claim 1, wherein the suction button is positioned on a first side of the device handle, and the suction inlet is positioned on a second side of the device handle, the second side of the device handle being adjacent to the first side of the device handle.

9. The bronchoscope device of claim 1, wherein the device handle comprises a t-connector or y-connector that joins a pressurized gas conduit and a suction conduit to the fluid channel.

10. A method of conducting a bronchoscopy operation on a patient comprising inserting an insertion tube of a bronchoscope device into a patient, the bronchoscopy device comprising:
insertion tube, the insertion tube comprising a fluid channel and a fiber optic element;
a device handle connected to the insertion tube, the handle comprising:
a pressurized gas inlet;
a pressurized gas button, the pressurized gas button has a pressurized gas off-state wherein pressurized gas is not supplied to the insertion tube and a pressurized gas on-state wherein pressurized gas is supplied to the insertion tube, the pressurized gas button being naturally bias in the pressurized gas off-state;
a suction inlet; and
a suction button, the suction button has a suction off-state wherein suction is not supplied to the insertion tube and a suction on-state wherein suction is supplied to the insertion tube, the suction button being naturally bias in the suction off-state;
wherein the user of the bronchoscope can supply: (1) pressurized gas to the insertion tube by pressing the pressurized gas button, and (2) suction to the insertion tube by pressing the suction button.

11. The method of claim 10, wherein the pressurized gas comprising oxygen is delivered through the insertion tube when the patient's SpO₂ falls below 90%.

12. The method of claim 11, wherein the insertion tube is not removed from the patient while the pressurized gas is being delivered.

13. The method of claim 11, wherein the bronchoscopy procedure is continued without removing the insertion tube after the patient's oxygen saturation is restored.

14. The method of claim 10, wherein the pressurized gas is medical grade oxygen.

15. The method of claim 10, wherein the pressurized gas inlet and the suction inlet are on the same side of the device.

16. The method of claim 10, wherein the selector is a button, and the pressurized gas inlet is integral with the button.

17. The method of claim 10, wherein the pressurized gas can be used to clear debris from the optical element at the distal end of the insertion tube during surgery.

18. The bronchoscope device of claim 10, wherein the pressurized gas button comprises a spring to bias the pressurized gas button in the pressurized gas off-state.

19. The bronchoscope device of claim 10, wherein the suction button comprises a spring to bias the suction button in the suction off-state.

20. The bronchoscope device of claim 10, wherein the pressurized gas inlet is attached to the pressurized gas button.

21. The bronchoscope device of claim 10, wherein the suction button is positioned on a first side of the device handle, and the suction inlet is positioned on a second side of the device handle, the second side of the device handle being adjacent to the first side of the device handle.

22. The bronchoscope device of claim 10, wherein the device handle comprises a t-connector that joins a pressurized gas conduit and a suction conduit to the fluid channel.
